(19) United States
(12) Patent Application Publication
(10) Pub. No.: US 2006/0293250 A1
(43) Pub. Date: Dec. 28, 2006

(54) Crystalline sugar compositions and method of making

(76) Inventors: Michael Major, Mequon, WI (US); Robert Peterson, Germantown, WI (US); Daniel Linse, Milwaukee, WI (US); Szymon Kosinski, Menomonee Falls, WI (US); Xiaoxiang Zhu, Cranbury, NJ (US)

Correspondence Address: BAKER & BOTTS, 30 ROCKEFELLER PLAZA, 44TH FLOOR, NEW YORK, NY, 10112-4498, US

(21) Appl. No.: 11/449,515

(22) Filed: Jun. 8, 2006

Related U.S. Application Data
(60) Provisional application No. 60/689,119, filed on Jun. 8, 2005.

(51) Int. Cl. A61K 31/7024; C07H 13/02; C07H 5/04

(52) U.S. Cl. 514/23; 536/119; 536/18.7

(57) ABSTRACT

Described are novel crystalline pivaloyl furanoses and methods of crystallizing the pivaloyl furanoses. These compounds are useful as intermediates in the synthesis of compounds such as the deoxyjirimycins and nojirimycins and are particularly useful as intermediates for production on a multi-kg scale. Particular crystalline compounds include 1,2,3,6-tetrapivaloyl-α-D-galactofuranose, 1,2,3,6-tetrapivaloyl-α-L-altrofuranose, and 5-azido-5-deoxy-1,2,3,6-tetrapivaloyl-α-D-galactofuranose.

### SPECIFICATION

This application claims priority from U.S. Provisional Patent Application Ser. No. 60/689,119, filed on Jun. 8, 2005, the disclosure of which is herein incorporated by reference in its entirety.

### FIELD OF THE INVENTION

This invention relates to crystalline pivaloyl furanoses and methods of crystallization of pivaloyl furanoses. These compounds are useful as intermediates in the synthesis of sugars such as D-1-deoxygalactonojirimycin (DGJ).

### BACKGROUND OF THE INVENTION

DGJ is also described as (2R,3S,4R,5S)-2-hydroxymethyl-3,4,5-trihydroxypiperidine, 1-deoxy-galactostatin and as D-1-deoxygalactonojirimycin. It is an iminosugar (5-amino-5-deoxy-D-glucopyranose) analogue of D-galactose, and is a potent inhibitor of both α- and β-D-galactosidases. Galactosidases catalyze the hydrolysis of glycosidic linkages and are important in the metabolism of complex carbohydrates. Galactosidase inhibitors such as DGJ can be used in the treatment of many diseases and conditions, including diabetes (e.g., U.S. Pat. No. 4,634,765), cancer (e.g., U.S. Pat. No. 5,250,545), herpes (e.g., U.S. Pat. No. 4,957,926), HIV and Fabry Disease (Fan et al., Nat. Med. 1999 5:1, 112-5).

The published chemical syntheses of nojirimycin derivatives such as deoxynojirimycin generally have multiple steps which are not suitable for commercial applications. Many of the intermediates are not stable, and purification of both the intermediates and the final products are unwieldy on a multi-kilogram scale. The chemo-microbiological method patented by Grabner (U.S. Pat. Nos. 5,695,969; 5,610,039) provides a method for transforming a sugar into its imino-derivative by reductive animation of a 5-keto aldose obtained by bacterial oxidation of glucose. The method is, however, not applicable to the D-galacto nojirimycin derivatives. Other related patents (U.S. Pat. Nos. 5,227,479, 4,908,439 and 4,634,765) discuss the preparation of homonojirimycin glycosides using protected glycosyl halides, hydride reduction of a D-glucuronolactone. U.S. Pat. No. 4,908,439 teaches a process of preparing glucose jirimycin derivatives, 5-amino-5-deoxy- 1,2-O-isopropylidene-D-gluconeurolactone (DNJ derivatives) by reacting an azide with a hydride reducing agent such as lithium aluminum hydride.

U.S. Pat. Nos. 6,740,780, 6,683,185, 6,653,482, 6,653,480, 6,649,766, 6,605,724, 6,590,121, and 6,462,197 describe a process for the preparation of imino sugars which are useful as intermediates in the preparation of D-dideoxy galacto nojirimycins. These compounds are 1,5-dideoxy-1,5-imino hexitols of a hexose sugars and are prepared from hydroxyl protected oxime intermediates. The process for making these imino sugars includes formation of a lactam which is reduced to the hexitol. However, this process has some disadvantages for production on a multi-kilogram scale with regard to safety, up-scaling, handling and synthesis complexity. For example, the process uses flash chromatography for purification, a procedure that is not practicable on large scale.

There are several preparations for D-1-deoxygalactonojirimycin (DGJ) published in the literature, most of which are not suitable for repetition in an industrial laboratory on a preparative scale procedure (>100 g). Some of these syntheses include a synthesis from D-glucose (Legler G, et al., Carbohydr Res. 1986 Nov 1;155:119-29); D-galactose (Uriel, C., Santoyo-Gonzalez, F., et al., Synlett 1999 593-595; Synthesis 1998 1787-1792 (disclosing pivaloylated intermediates); galactopyranose (Bernotas R C, et al., Carbohydr Res. 1987 Sep 15;167:305-11); L-tartaric acid (Aoyagi et al., J. Org. Chem. 1991, 56, 815); quebrachoitol (Chida et al., J. Chem. Soc., Chem Commun. 1994, 1247); galactofuranose (Paulsen et al., Chem. Ber. 1980, 113, 2601); benzene (Johnson et al., Tetrahedron Lett. 1995, 36, 653); arabino-hexos-5-ulose (Barili et al., tetrahedron 1997, 3407); 5-azido-1,4-lactones (Shilvock et al., Synlett, 1998, 554); doxynojirimicin (Takahashi et al, J Carbohydr. Chem. 1998, 17, 117); acetylglucosamine (Heightman et al., Helv. Chim. Acta 1995, 78, 514); myo-inositol (Chida N, et al., Carbohydr Res. 1992 Dec. 31;237:185-94); dioxanylpiperidene (Takahata et al., Org. Lett. 2003; 5(14); 2527-2529); and (E)-2,4-pentadienol (Martin R, et al., Org Lett. 2000 January;2(1):93-5) (Hughes A B, et al., Nat Prod Rep. 1994 April;11(2):135-62). A synthesis of N,N-methyl-1-deoxynojirimycin-containing oligosaccharides is described by Kiso (Bioorg Med Chem. 1994 November; 2(11):1295-308). Kiso coupled protected 1-deoxynojirimycin derivatives with methyl-1-thioglycosides (glycosyl donors) of D-galactose with a triflate used as the glycosyl promoter.

Although the use of column chromatography for purification is feasible for small scale synthesis, such as produced in the reactions taught by the references disclosed hereinabove, it is not sufficient for use on the multi-kg scale. The size of the column necessary as well as the quantity of solvents required makes this procedure impractical. The largest scale of DGJ prepare, as reported in the literature, is 13.3 g (see Fred-Robert Heiker, Alfred Matthias Schueller, Carbohydrate Research, 1989, 203 314-318), which is much less than is required for plant-scale synthesis for use as a therapeutic. Heiker et al. purified DGJ using the ion-exchange resin Lewatit MP 400 (OH⁻) and crystallization from ethanol. However, this process also cannot be readily scaled to multi-kilogram quantities.

Therefore, a synthesis which does not employe chromatography or ion exchange resins is preferred. The easiest method of isolating compounds in chemical manufacturing is crystallization. It is generally faster, safer, more cost-saving, and easier for scale-up then other methods. However, carbohydrates are usually in the form of oils, which are difficult to crystallize. There are some exceptions. For instance, U.S. Pat. No. 6,620,921 teaches crystalline 1,2,3,5,6-penta-O-propanoyl-β-D-glucofuranose, a compound useful for the preparation of some glucofuranosides. Although many glucofuranose derivatives are oils at normal temperatures and pressures, the '921 patent discloses that some furanoses are crystalline under these conditions. These firanoses include: phenyl β-D-glucofuranoside, 4-nitrophenyl α-D-glucofuranoside, methyl 2,3,5,6-tetra-O-propanoyl-1-thio-β-D-glucofuranoside, and 1-β D-glucofuranosyluracil.

However, there is still a need for other crystalline intermediates and for an easy, scaleable process for purifying the intermediates by crystallization, which is useful for the synthesis of deoxyjirimycins such as DGJ and is practical for large scale synthesis (including purifying the intermediates in the synthesis).

### SUMMARY OF THE INVENTION

Crystalline forms of furanoses and methods of crystallizing these furanoses are disclosed. The crystalline furanoses have at least one methylacetyl, dimethylacetyl, trimethylacetyl, or a protecting group.

The molecular weight of the furanose is between 300 g/mol and 1000 g/mol. Preferably, the molecular weight is at least 350 g/mol, at least 400 g/mol, or more preferably, at least 450 g/mol. In another embodiment, the molecular weight is less than 900 g/mol or less than 800 g/mol.

In another embodiment, there are at least three trimethylacetyl protecting groups. The furanose may be a tetrapivaloyl furanose such as 1,2,3,6-tetrapivaloyl-α-D-galactofuranose, 1,2,3,6-tetrapivaloyl-α-L-altrofuranose, or 5-azido-5-deoxy-1,2,3,6-tetrapivaloyl-α-D-galactofuranose.

Also provided is a method for producing a crystalline furanose comprising: adding the furanose to, or forming the furanose in, a solvent; and crystallizing the furanose from the solvent. The crystallization is preferably done by adding a second solvent and cooling at ambient pressure.

In one aspect of the present invention encompassing a crystalline tetrapivaloyl furanose, where least one of monopivaloyl, dipivaloyl, tripivaloyl, or pentapivaloyl furanose is formed in addition to the tetrapivaloyl furanose; this monopivaloyl, dipivaloyl, tripivaloyl, or pentapivaloyl furanose is not crystallized when the tetrapivaloyl furanose is crystallized. Similarly, where a tripivaloyl (or, e.g., pentapivaloyl or other protected sugar) is the intended product, and where additional unwanted protected sugars are formed in the reaction, the tripivaloyl (or, e.g., pentapivaloyl or other protected sugar) is crystallized from a solvent and the unwanted protected sugars are not.

Preferred solvents are heptane and methanol. In yet another aspect of the present invention, the crystallizing comprises heating the furanose and the solvent to a temperature near the boiling point of the solvent, cooling to a temperature below 0° C. or more preferably between −20° C. and −10° C., and waiting until the furanose precipitates; in one embodiment, this time is at least 36 hours.

In yet another embodiment the method of producing a crystalline furanose comprises: preparing a solution comprising a furanose and a first solvent; adding a second solvent, wherein the second solvent is miscible with the first solvent and capable of dissolving the furanose; and subjecting the solution to a crystallization treatment, to obtain said crystalline form of the furanose. The crystallization treatment may include cooling the solvent system, allowing the solution to cool without an external cooling source, waiting for a period of time with the solution at room temperature, adding a seed crystal, and/or adding an additional solvent or solvent system to cause the furanose to precipitate out of solution.

In yet another embodiment the method of producing a crystalline furanose comprises: preparing a solution comprising a furanose and one or more solvents, and slowly adding excess of an additional solvent, wherein the additional solvent is miscible with the first solvent and does not dissolve the furanose to obtain said crystalline form of the furanose.

In yet another embodiment, the present invention provides an improvement in a method of making nojirimycin derivatives such as DGJ. Such methods can be found, for example, in Santoyo-Gonzalez, F., et al., Synlett 1999 593-595. The improvement comprising crystallizing a furanose having least one methylacetyl, dimethylacetyl, trimethylacetyl, or other protecting group and using the furanose, without a purification step involving chromatography or ion exchange resin to purify the furanose, in the production of a nojirimycin derivative.

Other features, advantages and embodiments of the invention will be apparent to those skilled in the art from the following description, accompanying data and appended claims.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein.

FIG. 1. Synthesis of DGJ using crystalline derivatives II, III and IV.

FIG. 2. Synthesis of L-altrose using crystalline derivatives II and III.

FIG. 3. Synthesis of (2S,3S,4R,5S)-2-hydroxymethyl-piperidine-3,4,5-triol from D-galactose using crystalline derivatives II and V.

FIG. 4. Synthesis of (2R,3R,4S,5R,6R)-6-Hydroxymethyl-tetrahydro-thiopyran-2,3,4,5-tetraol (D-galactothiopyranose).

FIG. 5A. Proton NMR of crystallized 1,2,3,6-tetra-O-pivaloyl-α-D-galactofuranose (II), from 0 to 14 ppm.

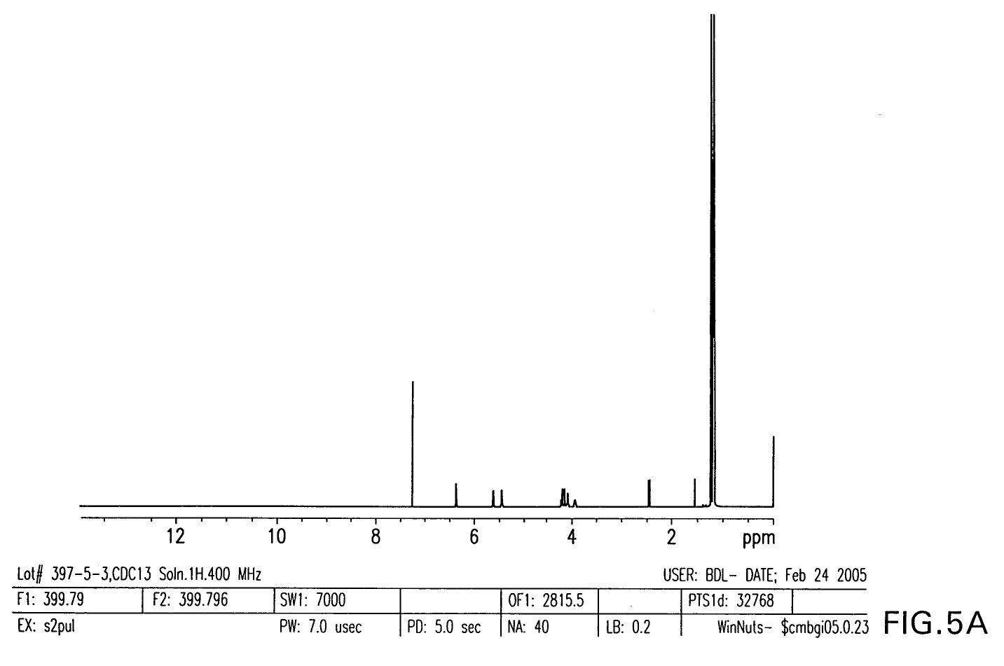

FIG. 5B. Proton NMR of crystallized 1,2,3,6-tetra-O-pivaloyl-α-D-galactofuranose (II), from 0.7 to 2.6 ppm.

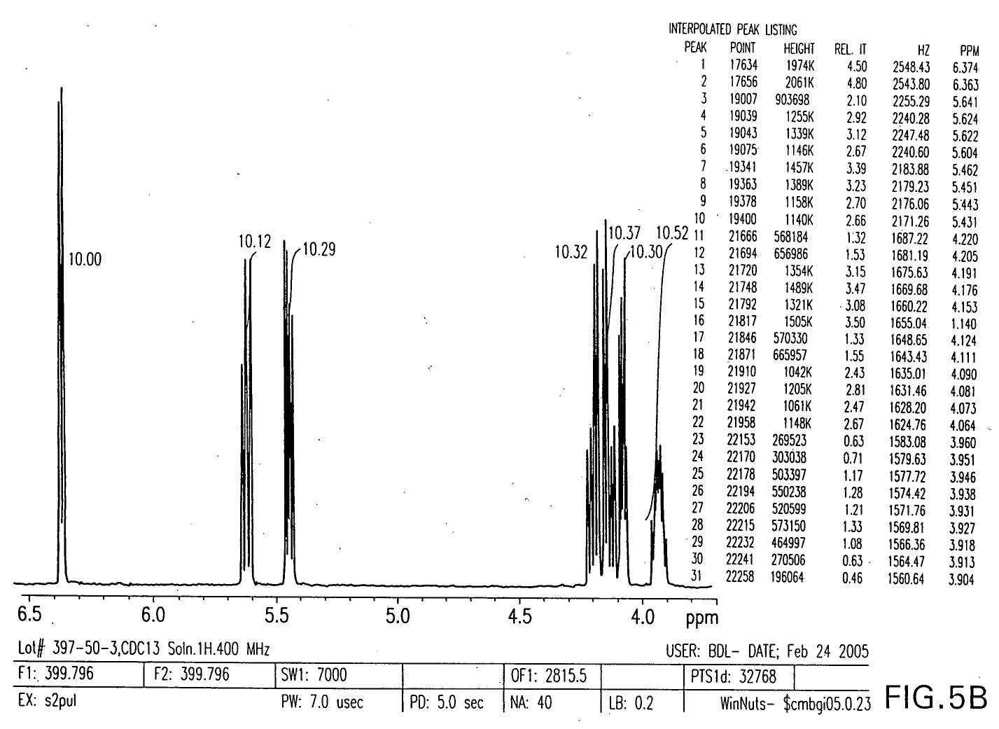

FIG. 5C. Proton NMR of crystallized 1,2,3,6-tetra-O-pivaloyl-α-D-galactofuranose (II), from 3.8 to 6.5 ppm.

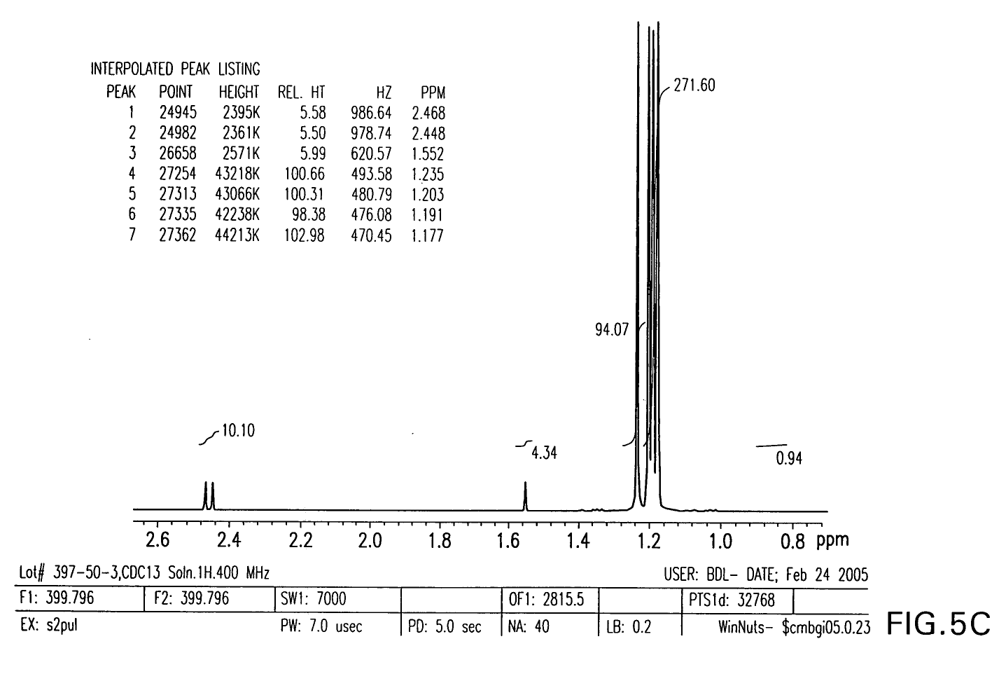

FIG. 6. HPLC of crystallized 1,2,3,6-tetrapivaloyl-α-L-altrofuranose (III)—showing complete removal of other isomers (II). Compound (III) elutes at approx. 27.5 min while the related isomer (II) would elute at 29.0 min.

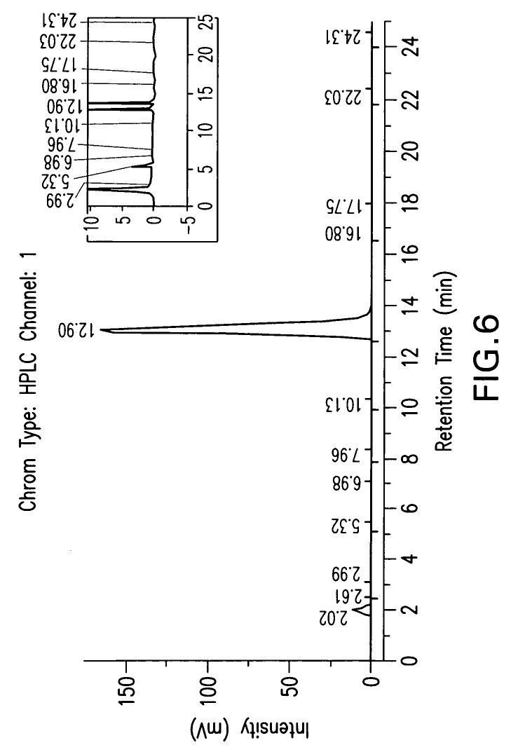

FIG. 7A. Proton NMR of crystallized 1,2,3,6-tetra-O-pivaloyl-α-L-altrofuranose, from 0 to 14 ppm.

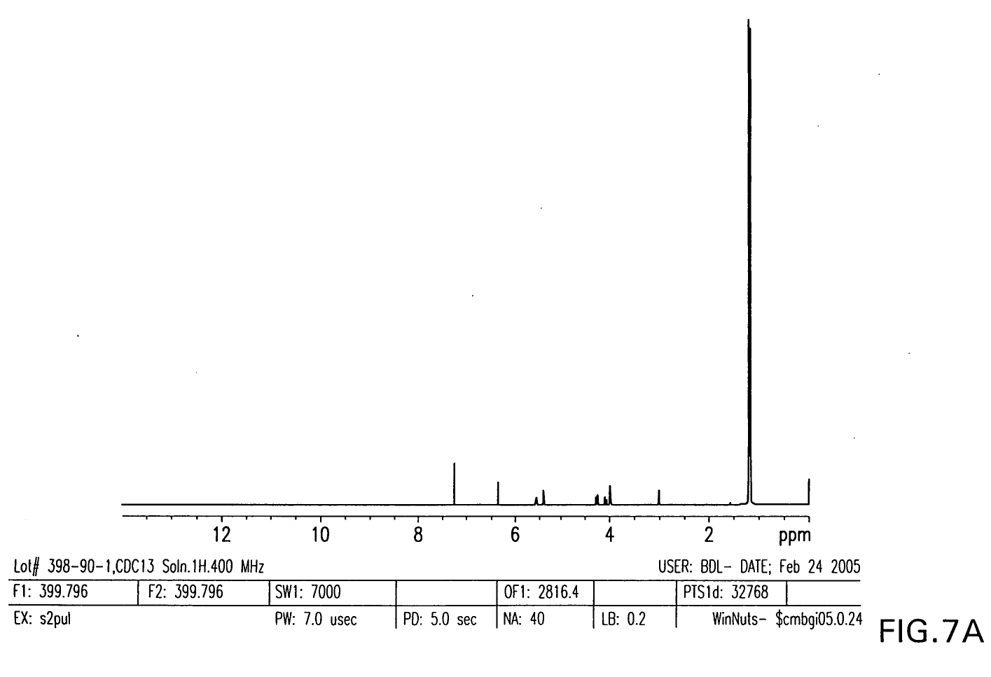

FIG. 7B. Proton NMR of crystallized 1,2,3,6-tetra-O-pivaloyl-α-L-altrofuranose from 3.8 to 6.6 ppm.

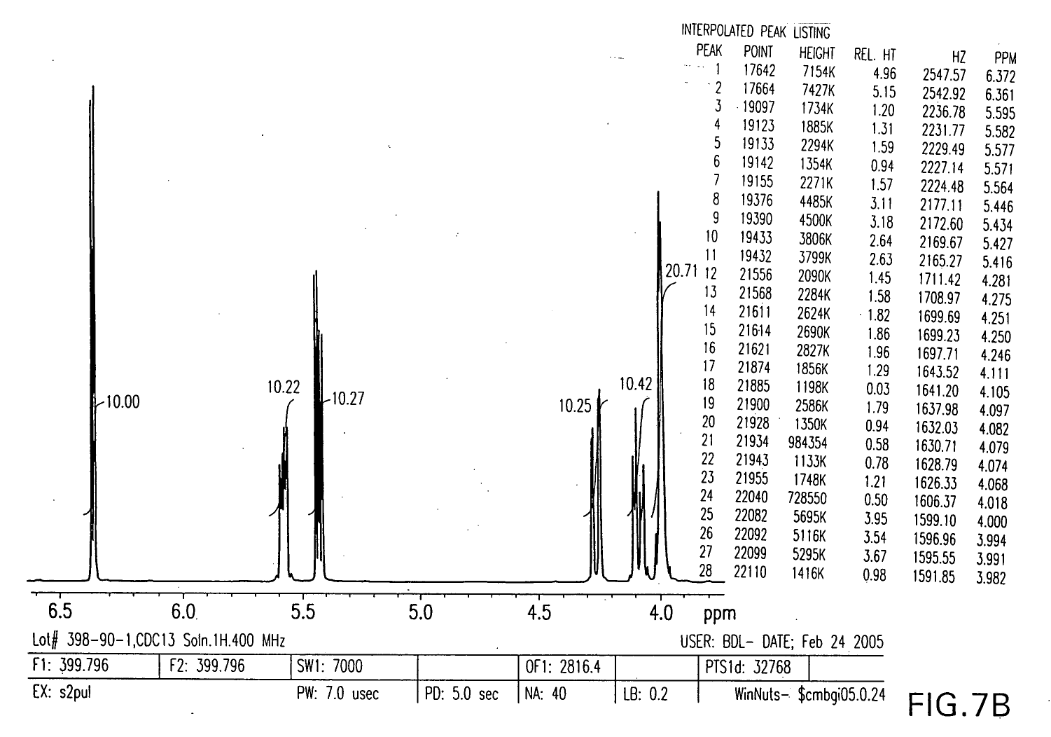

FIG. 7C. Proton NMR of crystallized 1,2,3,6-tetra-O-pivaloyl-α-L-altrofuranose from 0.7 to 3.2 ppm.

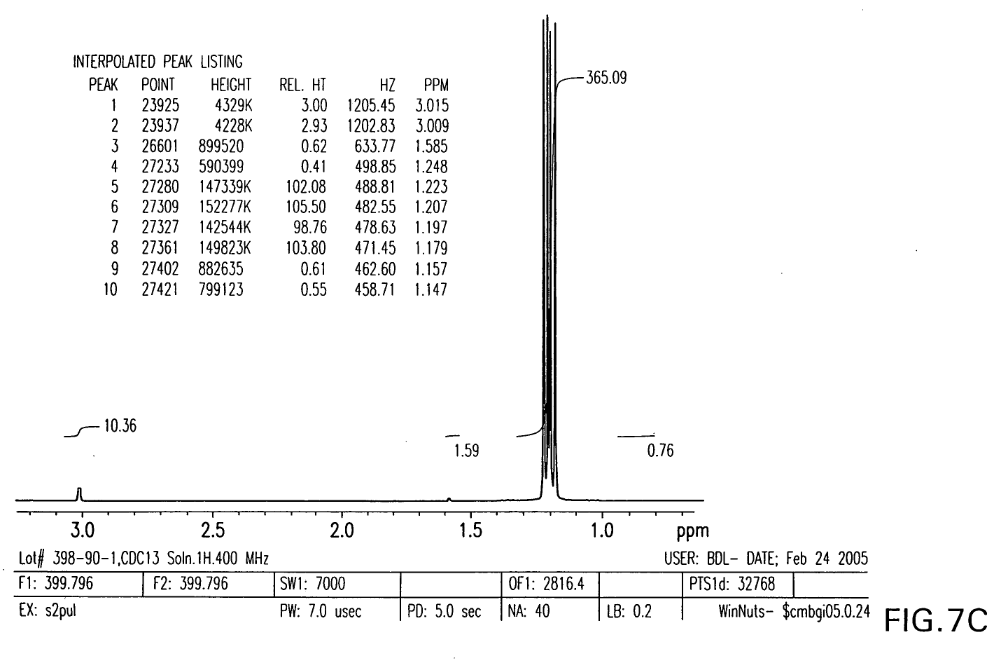

FIG. 8A. Proton NMR of crystallized 5-azido-5-deoxy-1,2,3,6-tetra-O-pivaloyl-α-L-altrofuranose, from 0 to 14 ppm.

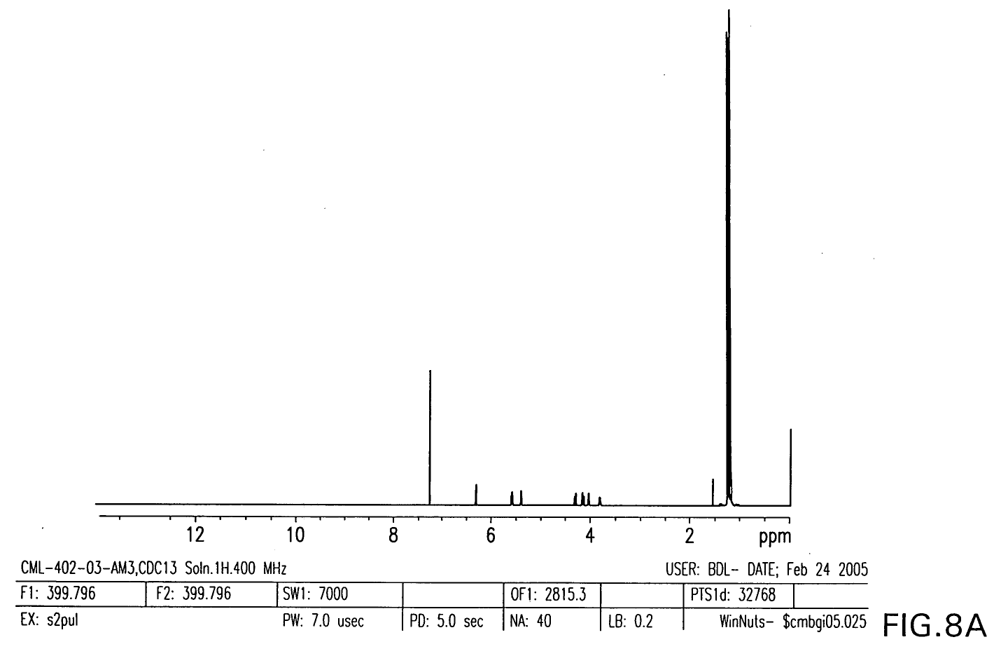

FIG. 8B. Proton NMR of crystallized 5-azido-5-deoxy-1,2,3,6-tetra-O-pivaloyl-α-L-altrofuranose, from 3.7 to 6.6 ppm.

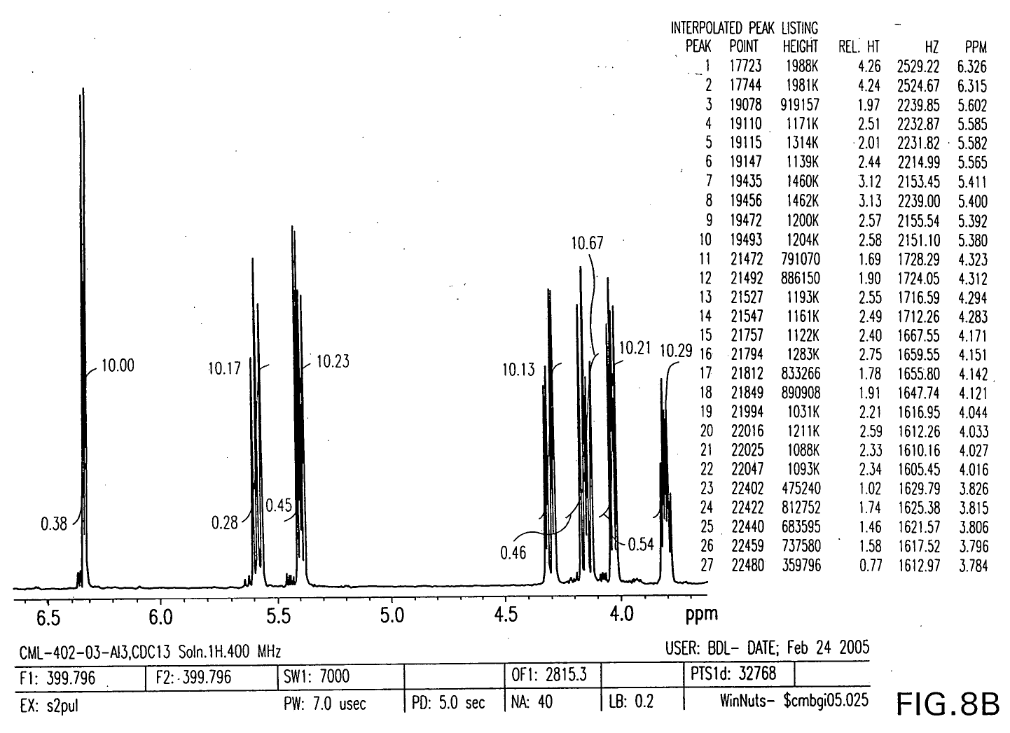

FIG. 8C. Proton NMR of crystallized 5-azido-5-deoxy-1,2,3,6-tetra-O-pivaloyl-α-L-altrofuranose, from 0.7 to 2.7 ppm.

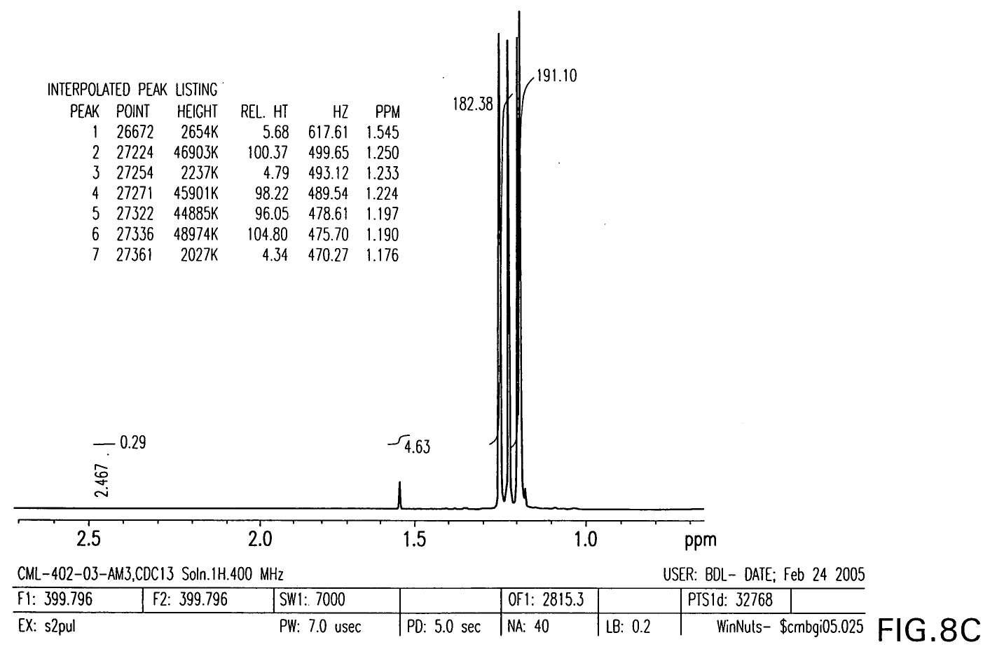

### DETAILED DESCRIPTION OF PREFERRED EMBODIMENTS

The term ‘alkyl’ refers to a straight or branched C1-C20 hydrocarbon group consisting solely of carbon and hydrogen atoms, containing no unsaturation, and which is attached to the rest of the molecule by a single bond, e.g., methyl, ethyl, n-propyl, 1-methylethyl (isopropyl), n-butyl, n-pentyl, 1,1-dimethylethyl (t-butyl). The alkyls used herein are preferably C1-C8 alkyls.

The term “alkenyl” refers to a C2-C20 aliphatic hydrocarbon group containing at least one carbon-carbon double bond and which may be a straight or branched chain, e.g., ethenyl, 1-propenyl, 2-propenyl (allyl), iso-propenyl, 2-methyl-1-propenyl, 1-butenyl, 2-butenyl.

The term “cycloalkyl” denotes an unsaturated, non-aromatic mono- or multicyclic hydrocarbon ring system such as cyclopropyl, cyclobutyl, cyclopentyl, cyclohexyl. Examples of multicyclic cycloalkyl groups include perhydronapththyl, adamantyl and norbornyl groups bridged cyclic group or sprirobicyclic groups, e.g., spiro (4,4) non-2-yl.

The term “cycloalkalkyl” refers to a cycloalkyl as defined above directly attached to an alkyl group as defined above, which results in the creation of a stable structure such as cyclopropylmethyl, cyclobutylethyl, cyclopentylethyl.

The term “alkyl ether” refers to an alkyl group or cycloalkyl group as defined above having at least one oxygen incorporated into the alkyl chain, e.g., methyl ethyl ether, diethyl ether, tetrahydrofuran.

The term “alkyl amine” refers to an alkyl group or a cycloalkyl group as defined above having at least one nitrogen atom, e.g., n-butyl amine and tetrahydrooxazine.

The term “aryl” refers to aromatic radicals having in the range of about 6 to about 14 carbon atoms such as phenyl, naphthyl, tetrahydronapthyl, indanyl, biphenyl.

The term “arylalkyl” refers to an aryl group as defined above directly bonded to an alkyl group as defined above, e.g.,—CH2C6H5, and —C2H4C6H5.

The term “heterocyclic” refers to a stable 3- to 15-membered ring radical which consists of carbon atoms and from one to five heteroatoms selected from the group consisting of nitrogen, phosphorus, oxygen and sulfur. For purposes of this invention, the heterocyclic ring radical may be a monocyclic, bicyclic or tricyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized; and the ring radical may be partially or fully saturated (i.e., heteroaromatic or heteroaryl aromatic). Examples of such heterocyclic ring radicals include, but are not limited to, azetidinyl, acridinyl, benzodioxolyl, benzodioxanyl, benzofurnyl, carbazolyl, cinnolinyl, dioxolanyl, indolizinyl, naphthyridinyl, perhydroazepinyl, phenazinyl, phenothiazinyl, phenoxazinyl, phthalazinyl, pyridyl, pteridinyl, purinyl, quinazolinyl, quinoxalinyl, quinolinyl, isoquinolinyl, tetrazoyl, imidazolyl, tetrahydroisouinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazinyl, pyrimidinyl, pyridazinyl, oxazolyl, oxazolinyl, oxasolidinyl, triazolyl, indanyl, isoxazolyl, isoxasolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzooxazolyl, furyl, tetrahydrofurtyl, tetrahydropyranyl, thienyl, benzothienyl, thiamorpholinyl, thiamorpholinyl sulfoxide thiamorpholinyl sulfone, dioxaphospholanyl, oxadiazolyl, chromanyl, isochromanyl.

The heterocyclic ring radical may be attached to the main structure at any heteroatom or carbon atom that results in the creation of a stable structure.

The term “heteroaryl” refers to a heterocyclic ring wherein the ring is aromatic.

The term “heteroarylalkyl” refers to heteroaryl ring radical as defined above directly bonded to alkyl group. The heteroarylalkyl radical may be attached to the main structure at any carbon atom from alkyl group that results in the creation of a stable structure.

The term “heterocyclyl” refers to a heterocylic ring radical as defined above. The heterocyclyl ring radical may be attached to the main structure at any heteroatom or carbon atom that results in the creation of a stable structure.

The term “heterocyclylalkyl” refers to a heterocylic ring radical as defined above directly bonded to alkyl group. The heterocyclylalkyl radical may be attached to the main structure at carbon atom in the alkyl group that results in the creation of a stable structure.

The substituents in the ‘substituted alkyl’, ‘substituted alkenyl’ ‘substituted alkynyl’ ‘substituted cycloalkyl’ ‘substituted cycloalkalkyl’ ‘substituted cycloalkenyl’ ‘substituted arylalkyl’ ‘substituted aryl’ ‘substituted heterocyclic ring’, ‘substituted heteroaryl ring,’ ‘substituted heteroarylalkyl’, or ‘substituted heterocyclylalkyl ring’, may be the same or different with one or more selected from the groups hydrogen, hydroxyl, halogen, carboxyl, cyano, amino, nitro, oxo (═O), thio (═S), or optionally substituted groups selected from alkyl, alkoxy, alkenyl, alkynyl, aryl, arylalkyl, cycloalkyl, aryl, heteroaryl, heteroarylalkyl, heterocyclic ring, —COORx, —C(O)Rx, —C(S)Rx, —C(O)NRxRy, —C(O)ONRxRy, —NRxCONRyRz, —N(Rx)SORy, —N(Rx)SO2Ry, —(═N—N(Rx)Ry), —NRxC(O)ORy, —NRxRy, —NRxC(O)Ry—, —NRxC(S)Ry —NRxC(S)NRyRz, —SONRxRy—, —SO2NRxRy—, —ORx, —ORxC(O)NRyRz, —ORxC(O)ORy—, —OC(O)Rx, —OC(O)NRxRy, —RxNRyRz, —RxRyRz, —RxCF3, —RxNRyC(O)Rz, —RxORy, —RxC(O)ORy, —RxC(O)NRyRz, —RxC(O)Rx, —RxOC(O)Ry, —SRx, —SORx, —SO2Rx, —ONO2, wherein Rx, Ry and Rz in each of the above groups can be hydrogen atom, substituted or unsubstituted alkyl, haloalkyl, substituted or unsubstituted arylalkyl, substituted or unsubstituted aryl, substituted or unsubstituted cycloalkyl, substituted or unsubstituted cycloalkalkyl substituted or unsubstituted heterocyclic ring, substituted or unsubstituted heterocyclylalkyl, substituted or unsubstituted heteroaryl or substituted or unsubstituted heteroarylalkyl.

The term “halogen” refers to radicals of fluorine, chlorine, bromine and iodine.

It has been found that pivaloyl furanose compounds can be readily obtained in crystalline form. The purification of these compounds by crystallization is simplified relative to the purification of non-crystalline products, especially on large scale synthesis where purification by chromatography is not feasible. Although chromatography can be a useful tool, it is ineffective in multi-kilogram scale syntheses. The pivaloyl furanoses produced by the methods of the present invention are useful in the synthesis of sugars, and are particularly relevant for synthesis processes where purification by chromatography is inappropriate. A large variety of sugars and derivatives of sugars can be made by the crystallization methods described herein, since the protected furanose compounds can be stereoselectively synthesized and isolated by crystallization. For example, sugars such as L-altrose can be made from the less expensive sugars such as D-galactose sugars by first creating a selectively pivaloylated intermediate, inversion of the configuration at carbon C-5, purifying the intermediate by crystallization, and then deprotecting to form the sugar. Compounds such as D-1-deoxygalactonojirimycin (DGJ) can be made using the pivaloyl furanoses of the current invention. The crystalline pivaloyl furanoses are useful intermediates in the synthesis of DGJ which can be purified by crystallization without the use of chromatographic separation, to allow for the multi-kilogram scale synthesis with high purity and good yields.

Sugars

The current invention allows for the isolation of crude protected furanoses by decanting the solution from the solid, crystalline product formed in the reaction. This is preferred over the isolation methods found in the literature due to the simplicity and reduced cost compared to column chromatography and other methods. This is possible because of the surprising finding that the pivaloyl furanoses may be crystallized and isolated as solids.

The furanose compounds that may be purified by the method described herein include the protected furanose compounds with a molecular weight of greater than 300 g/mol having the formula: 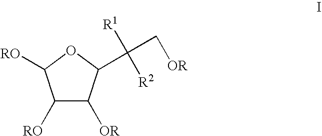
wherein each R is independently H, acetyl, methylacetyl, dimethylacetyl, trimethylacetyl, or a protecting group, and at least two Rs are selected from the group consisting of methylacetyl, dimethylacetyl, and trimethylacetyl. In a preferred embodiment, each R is trimethylacetyl (pivaloyl). In anther embodiment, the sugar has three pivaloyl groups.

R¹ and R² are H, OH, OR³, N₃, NH₂, NHR³, NR³₂, SH, SR³, OS(═O)₂R³, C(═O)R³, methylacetoxy, dimethylacetoxy, trimethylacetoxy, acetoxy, chloroacetoxy, dichloroacetoxy, trichloroacetoxy or an O-protecting group, wherein at least one of R¹ and R² is H. Each R³ is independently H or a substituted or unsubstituted C₁-C₁₂ alkyl, C₂-C₁₂ alkenyl, C₂-C₁₂ alkynyl, C₅-C₆ cycloalkyl, C₅-C₁₂ cycloalkenyl, C₅-C₁₂ aryl, C₄-C₁₂ heteroaryl, C₆-C₁₂ arylalkyl, C₄-C₁₂ heterocycle, C₆-C₁₂ heterocycloalkyl, C₅-C₁₂ heteroarylalkyl or a C₂-C₁₂ acyl. In one embodiment, each R is a pivaloyl and one of R¹ and R² is trimethylacetoxy (pentapivaloyl).

Preferred aryls and arylalkyls are phenyl, benzyl or C₇-C₁₂ alkylphenyl, especially C₁-C₄ alkylphenyl or alkylbenzyl. Preferred acyls are C₂-C₈ acyl, for example, acetyl, propanoyl, butanoyl, pentanoyl, hexanoyl and benzoyl. Preferred alkyls are C₁-C₆ alkyls.

Any of the positions having an OR moiety may be protected or left as an OH. The location of the free hydroxyl group is defined by regioselectivity of the performed reaction.

The R groups are selected such that the molecular weight of the furanose is at least 300 g/mol. Preferably, the molecular weight is at least 325 g/mol, or at least 350 g/mol, or at least 375 g/mol, or at least 400 g/mol, or at least 425 g/mol. Most preferably, the molecular weight is at least 500 g/mol. In some embodiments, the molecular weight will be at least 525 g/mol or 550 g/mol, or 575 g/mol, or 600 g/mol. The molecular weight will be less than 1000 g/mol, and preferably less than 800 g/mol.

A preferred protecting group is the pivaloyl group. This protecting group is large, having a molecular weight of 85 g/mol, and can be considered as a crystal maker as for example other very large group triphenylmethyl group. The large size allows the sugar moiety to crystallize instead of remaining oil, as would smaller compounds. Alternatively, dimethyl acetyls may be used as the protecting group. Although the acetyl group and methylacetyl are both too small to be crystal maker protecting groups, it is contemplated that one or two R groups may be acetyl or methylacetyl where the remaining R groups are dimethyl acetyl or trimethyl acetyl groups where the compound has a molecular weight of at least 300 g/mol.

For a compound having four pivaloyl groups (at 85 g/mol each), a sugar having a hydroxyl group at R₁ or R₂ position will have a molecular weight of 516 g/mol; if one of R₁ or R₂ is an azide, the molecular weight is 541 g/mol. Each of these compounds will crystallize from the appropriate solvent. Compounds with a molecular weight of at least 300 g/mol will also crystallize. Therefore, if instead of four pivaloyl groups, the sugar is protected using four dimethylacetyl groups, (for comparative molecular weights of 460 and 485 g/mol for the azo sugar) the sugar can be crystallized as described herein.

Similarly, for a compound having three pivaloyl groups, a sugar having two hydroxyl groups: at R₁ or R₂ position and elsewhere in the molecule will have a molecular weight of 432 g/mol; if one of R₁ or R₂ is an azide, the molecular weight is 457 g/mol. Each of these compounds will crystallize from the appropriate solvent.

In a preferred embodiment, tetrapivaloyl furanoses are crystallized. Since the protection reaction will potentially form mono, di-, tri-, and penta-pivaloyl derivatives as well as the desired tetra-pivaloyl derivatives (or alternatively, tetra pivaloyl will form in addition to the preferred penta-pivaloyl or tri-pivaloyl), crystallization of only the desired product allows for the separation of these side products/impurities. The solvent or solvent systems used can be ‘tuned’ to the particular tetrapivaloyl furanose to be crystallized based on the molecular weight and polarity of the compound.

It is contemplated that one or more of the protecting groups is not a pivaloyl or related alkylacetyl group. Other protecting groups that may be contained as part of the pivaloyl furanose of the current invention include detachable protective groups that derivatize the hydroxyl groups of sugar. For example, the furanose may contain four pivaloyl groups and one other protecting group, or three pivaloyl groups and two other protecting groups, or two pivaloyl groups and two other protecting groups, or three pivaloyl groups and one protecting group and one hydroxyl group. Protective groups of this type and processes for forming derivatives are generally known in sugar chemistry and include, but are not limited to: linear or branched C₁-C₈ alkyl, especially C₁-C₄ alkyl, for example, methyl, ethyl, n-propyl, isopropyl or n-, iso- and t-butyl; C₇-C₁₂ arylalkyl, for example, benzyl, trialkylsilyl having 3 to 20, particularly 3 to 10, C atoms, for example, trimethylsilyl, triethylsilyl, tri-n-propylsilyl, isopropyldimethylsilyl, t-butyldimethylsilyl, n-octyldimethylsilyl or (1,1,2,2-tetramethylethyl)-dimethylsilyl; substituted methylidene groups which are obtainable by forming acetals or ketals from adjacent OH groups of the sugars or sugar derivatives by means of aldehydes and ketones and which preferably contain 2 to 12, or 3 to 12, respectively, C atoms, for example, C₁-C₁₂ alkylidene, preferably C₁-C₆ alkylidene and particularly C₁-C₄ alkylidene, or benzylidene (ethylidene, 1,1-propylidene, 2,2-propylidene, 1,1-butylidene or 2,2-butylidene); C₂-C₁₂ acyl, especially C₂-C₈ acyl, for example, acetyl, propanoyl, butanoyl, pentanoyl, hexanoyl and benzoyl; R₅ —SO—, in which R₅ is C₁-C₁₂ alkyl, especially C₁-C₆ alkyl, C₅ cycloalkyl, C₆ cycloalkyl, phenyl, benzyl or C₇ -C₁₂ alkylphenyl, especially C₁-C₄ alkylphenyl, or C₁-C₁₂ alkylbenzyl, especially C₁-C₄ alkylsulfonyl or arylsulfonyl, for example, methylsulfonyl, ethylsulfonyl, propylsulfonyl, butylsulfonyl, phenylsulfonyl, benzylsulfonyl and p-methylphenylsulfonyl. (See, for example, U.S. Pat. No. 5,218,097). Preferred protecting groups that are used in addition to one or more pivaloylate group are acetyl, benzyl, silyl or trityl.

The sugar structure depicted herein is the hexofuranose form. However, the crystalline sugar may also conform to another form, such as the cyclic hemiacetal in either five- (as in furanose) or six-membered (as in pyranose) ring form and open chain form.

Other pivaloyl furanoses can be purified by the crystallization method of the invention. The furanosides may also be produced by the method described herein by using different starting material. For example, any one of the sugars: allose, altrose, glucose, mannose, gulose, idose and talose may be used as a starting material to produce crystalline pivaloyl furanoses. Both the D- and L-series of the furanose compounds described herein are contemplated; the more preferred stereochemistry comprises the D-series.

Crystallization

The compounds of the invention have been found to be crystalline and do not require the use of the purification procedure described in the literature, such as column chromatography or ion exchange resins as are commonly required during sugar synthesis since the sugars generally are in the form of viscous liquids and cannot be crystallized.

The furanose sugar may be crystallized by methods well known in the art. Solvents are chosen based on the polarity and lack of reactivity with the sugar. The ideal solvent for the crystallization must not react with the sugar, dissolve a moderately large amount of the furanose when hot and only a small amount of the furanose when cool. The solvent also should boil at temperature below the sugar's melting point. There are a number of solvents that may be used. In general, more polar sugars such as galactose and altrose sugars will crystallize from more non-polar solvents such as C₆- C₉ alkanes and cycloalkanes. Other sugars, such as those substituted with an azide, are less polar and a more polar solvent such as methanol should be used for crystallization.

Solvents that may be used in the current invention include, but are not limited to, ethanol, methanol, propanol, n-hexane, cyclohexane, heptane, octane, tetrahydrofuran, diethyl ether, ethyl acetate, dibutyl ether, dimethyl ether, diisopropyl ether, tert-butyl-methyl ether, methylene chloride, chloroform, carbon tetrachloride, dichloromethane, 1,2-dichloroethane, 1,1,2,2-tetrachloroethane, dioxane, acetonitrile, pentanol, isopropanol, benzene, toluene, xylene, acetone, ethylene glycol, and a combination of two or more of these solvents.

The amount of sugar solvated in the solvent when it is hot or boiling is preferably between 5 and 60% by weight. More preferably, there is 10-50%, or 20-40%, or most preferably, 25-35% sugar by weight in the solvent. After solvating the sugar, the temperature is reduced. Preferably the temperature is reduced to below 0° C., or more preferably to −10° C. or −20°C. for crystallization. If preferred, seeding may be used. The crystallization of the furanose proceeds slowly, which allows for the exclusion of impurities as the crystal structure grows, since the molecules in the crystal lattice are in equilibrium with the molecules in solution. In one embodiment, the solution is maintained at between −10° C. and −20° C. for about two days for crystallization to occur.

This invention provides furanose sugars having at least one methylacetyl, dimethylacetyl, trimethylacetyl, or a protecting group that are produced at a purity level greater, and preferably significantly greater, than can be achieved by other methods of making furanose sugars without resorting to the additional step of purification by column chromatography or ion exchange resin. These crystalline furanose sugars are substantially more pure. The crystallization process as described herein is advantageous since it allows for the separation of the furanose crystals from contaminants, including reaction byproducts having additional pivaloylate moieties, or unprotected groups.

In one embodiment, crude pivaloyl furanose is isolated by crystallization from a solution such as an aqueous DMF solution. This solution is useful since it can be used during the formation of the protected furanose and is obtained after quenching the protection reaction. The crystallization from DMF solution can take up to about 2 days. Once the crude product is collected, it is dissolved in solutions such as heptane/ethyl acetate. It can then be purified by washing, drying, concentrated and recrystallized from, e.g., heptane. This recrystallization process leaves contaminants and side products (such as penta-pivaloylate when tetrapivaloylate is desired) that were formed in the reaction in the mother liquor while the desired pivaloyl furanose is crystallized. This recrystallization is also slow and may take up to 2 days. Seeding also may be used in this reaction if desired.

In one preferred embodiment, the tetrapivaloyl furanose 1,2,3,6-tetra-O-pivaloyl-α-D-galactofuranose (II) or 1,2,3,6-tetrapivaloyl-α-L-altrofuranose (III) are isolated by crystallization from a C6-C9 alkane, such as hexane or heptane. These furanoside products can be produced with high purity. In another preferred embodiment, the azide tetrapivaloyl furanose 5-azido-5-deoxy-1,2,3,6-tetrapivaloyl-α-D-galactofuranose (IV) is isolated by crystallization from methanol. This affords a product with better purity than crystallization from heptane as performed with the tetrapivaloyl furanose compounds (II) and (III). Similar azido sugars, such as 5-azido-5-deoxy-1,2,3,6-tetrapivaloyl-α-L-altrofuranose, 5-azido-5-deoxy- 1,2,3,6-tetrapivaloyl-α-D-altrofuranose, and 5-azido-5-deoxy-1,2,3,6-tetrapivaloyl-α-L-galactofuranose are also contemplated.

### Synthesis of DGJ

In a synthesis method for DGJ, D-galactose can be used as a starting material, as described by Santoyo-Gonzalez (1999), incorporated herein by reference. The strategy in this synthesis includes: protection of the hydroxyl groups of D-galactose with pivaloyl groups by reacting the sugar with 1-(trimethylacetyl)imidazole (pivaloyl imidazole) in N,N-dimethylformamide (DMF) to form the protected furanoside derivatives: 1,2,3,6-tetra-O-pivaloyl-α-D-galactofuranose (II) as the major product and a mixture of the α,β-anomers of 1,2,3,5,6-penta-O-pivaloyl-D-galactofuranose as the minor ones. The galaoctofuranoside is then converted to the altrofuranoside, 1,2,3,6-tetrapivaloyl-α-L-altrofuranose (III). Next, the hydroxyl is protected and substituted with an azido group to obtain 5-azido-5-deoxy-1,2,3,6-tetrapivaloyl-α-D-galactofuranose (IV). After deprotecting, the galactofuranoside intermediate is reduced to obtain DGJ. Santoyo-Gonzalez used column chromatography to purify the three furanoside intermediates as well as the DJG product. The synthesis of DGJ described in this reference is only useful for a scale of about 200 mg final product with about 20% overall yield.

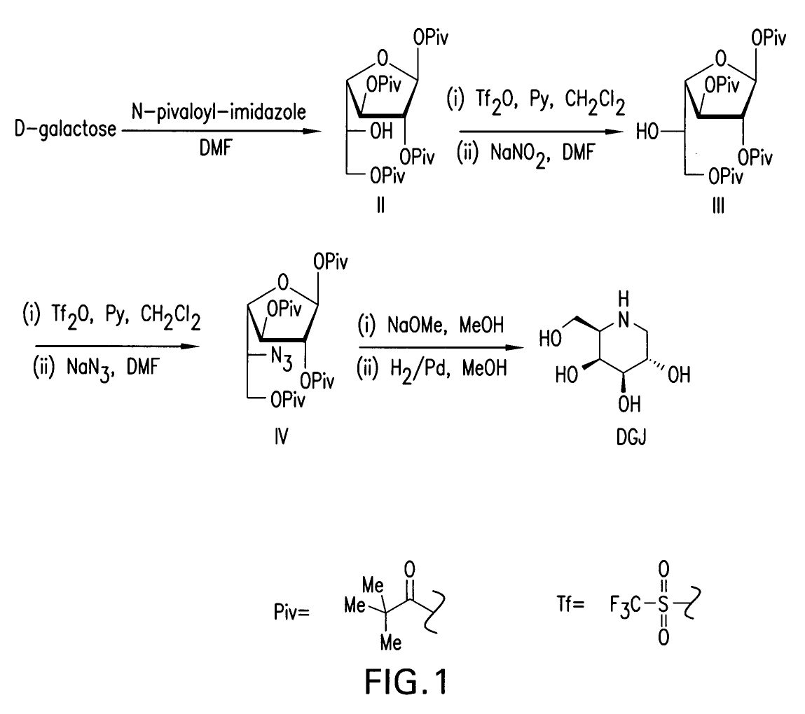

Since the three furanoside intermediates, 1,2,3,6-tetra-O-pivaloyl-α-D-galactofuranose (II), 1,2,3,6-tetrapivaloyl-α-L-altrofuranose (III), and 5-azido-5-deoxy-1,2,3,6-tetrapivaloyl-α-D-galactofuranose (IV) can each be crystallized from common solvents, the current invention provides an improved method of synthesis of DGJ (FIG. 1). Instead of using column chromatography for purification during each of the intermediate steps, the furanoside intermediates may be purified by crystallization. The galactofuranoside (IV) can be used to form DGJ, such as by the method described by Santoyo-Gonzalez.

### Synthesis of Altrose Derivatives

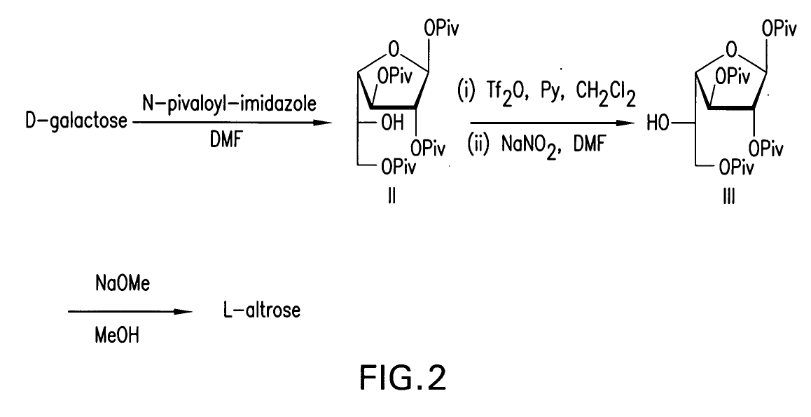

L-altrose is a nonnutritive sweetener which may be synthesized by a sequence of chemical reactions with low overall yields or from extracellular polysaccharides cultivated from the bacterium Butyrivibrio fibrisolvens (U.S. Pat. No. 4,966,845). However, these methods are expensive. Use of the crystalline pivaloyl furanoses of the current invention allows for the simple conversion of D-galactose derivatives to the more expensive L-altrose derivatives. This can be accomplished without the need for chromatographic separation and purification (FIG. 2). The crystalline 1,2,3,6-tetra-O-pivaloyl-α-L-altrofuranose can be prepared in the manner described above starting from inexpensive D-galactose. Later, the crystalline 1,2,3,6-tetra-O-pivaloyl-α-L-altrofuranose can undergo a deprotection reaction to remove pivaloyl protecting groups (e.g. sodium methoxide in methanol) and pure α-L-altrofuranoside can be isolated.

### Synthesis of other Sugars

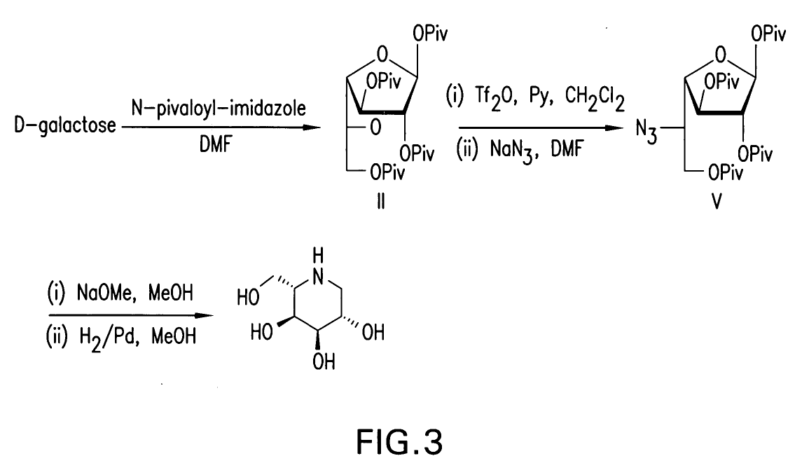

The pivaloyl furanoses of the current invention are useful intermediates in the synthesis of numerous sugars and sugar derivatives. For example, analogously to the synthesis of DGJ, D-galactose can be used as a starting material to prepare (2S,3S,4R,5S)-2-hydroxymethyl-piperidine-3,4,5-triol, as described by Santoyo-Gonzalez (1999), herein incorporated by reference (FIG. 3). The crystalline 1,2,3,6-tetra-O-pivaloyl-α-D-galactofuranose (II) can be prepared as described above. Next, the hydroxyl is protected and substituted with an azido group to obtain 5-azido-5-deoxy-1,2,3,6-tetrapivaloyl-α-L-altro-furanose (V). After deprotecting, the 5-azido altrofuranoside intermediate is reduced to obtain iminosugar. Since the furanoside intermediates, 1,2,3,6-tetra-O-pivaloyl-α-D-galactofuranose (II) and 5-azido-5-deoxy-1,2,3,6-tetrapivaloyl-α-L-altrofuranose (V) can each be crystallized from common solvents, the current invention provides an improved method of synthesis of (2S,3S,4R,5S)-2-hydroxymethyl-piperidine-3,4,5-triol - isomer of DGJ.

Thiohexoses, such as those described by Whistler, may also by pivaloylated and crystallized by the methods described herein. (Whistler, J. Org. Chem., 1968, 396-8).

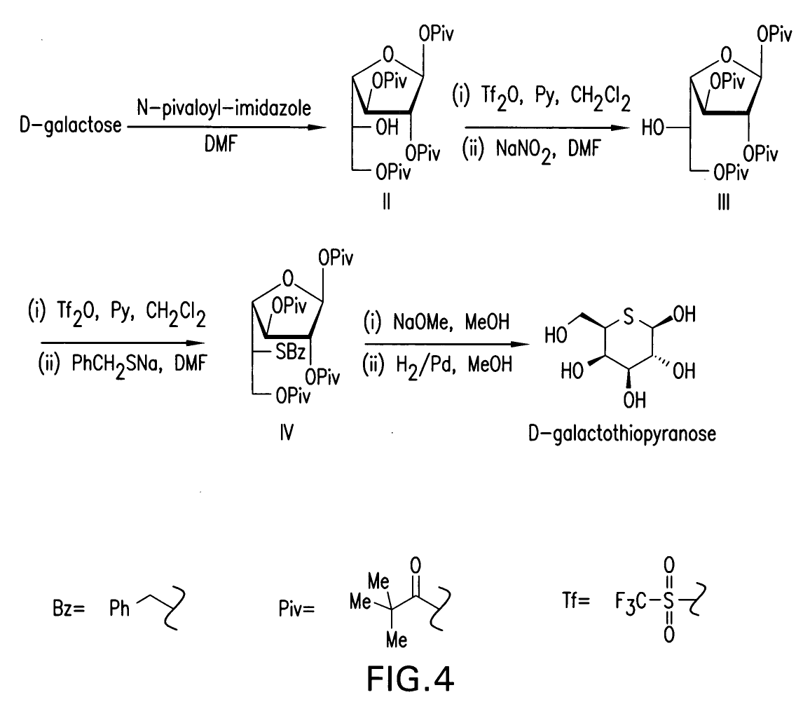

D-galactose can be used as a starting material to prepare (2R,3R,4S,5R,6R)-6-hydroxymethyl-tetrahydro-thiopyran-2,3,4,5-tetraol (D-galactothiopyranose) (FIG. 4), analogously to the synthesis of DGJ. 1,2,3,6-Tetrapivaloyl-α-L-altrofuranose (III) can be prepared as described above. The hydroxyls are protected and substituted with a benzylthio group to obtain 5-benzylthio-5-deoxy- 1,2,3,6-tetrapivaloyl-α-D-galactofuranose (IV). This tetrapivaloyl can be crystallized to purify this intermediate. After deprotecting, the galactofuranoside intermediate is reduced to obtain D-galactothiopyranose.

As used herein, the term “multi-kilogram,” multi-kg” and “preparatory scale” denote a scale of synthesis where the product is in an amount greater than one kg, or even more than 10 or more kg of product in a single synthesis.

### EXAMPLES

The present invention is further illustrated in the following examples, which should not be taken to limit the scope of the invention.

### Example 1

### Preparation and characterization of crystalline 1,2,3,6-tetrapivaloyl-α-D-galactofuranose (II)

1-(Trimethylacetyl)imidazole (pivaloyl imidazole) ( 42.2 kg, 5-fold excess) was dissolved in DMF (90 kg) and heptane (3.4 kg) and solution warmed to 60° C. D-Galactose (10 kg) was charged to the solution and mixture was heated to 75° C. The reaction was allowed to exotherm to 90-100° C. and after exotherm subsides the reaction was maintained at 80-100° C. until complete. The progress of the reaction was monitored by TLC (hexane:ethyl acetate =4:1). To visualize the progress, the TLC was later stained with dilute sulfuric acid and heated; the reaction was deemed complete when the spot of the product on TLC (R_{f}=0.5) became a major component. After the reaction was completed, the reaction product was immediately transferred to mixture containing water (200 kg) and ice (82 kg). Crude product was isolated by crystallization from this mixture; this crystallization was slow, generally taking two days. The crude product was collected and dissolved in heptane/ethyl acetate and washed with water, dried with magnesium sulfate, concentrated and crystallized again from 2-3 volumes of heptane (˜25 kg) at −20° C.; this process left the penta-pivaloylate in the mother liquor. The yield for this step was 25-35% (7.2-10 kg) when performed on a multi-kg scale. The 1,2,3,6-tetrapivaloyl-α-D-galactofuranose (II) was a white crystalline powder having high purity. Melting point was within the range of 105-108° C. IR (KBr, cm⁻¹): 3432 (OH, s), 2974 (C—H stretch, s), 1740 (ester of pivaloylate, vs), 1284 (C—O, weak), 1143 (C—O, vs), 1031 (C—O, weak); ¹H NMR (CDCl₃, 400 MHz, TMS): δ=1.18 (s, 3H), 1.19 (s, 3H), 1.20 (s, 3H), 1.23 (s, 3H), 2.45 (d, J=7.9 Hz, 1H); 3.90-3.96 (m, 1H), 4.07 (dd, J=3.4 Hz, J=6.5 Hz, 1H), 4.13 (dd, J=11.6 Hz, J=5.3 Hz, 1H), 4.19 (dd, J=11.6 Hz, J=6.1 Hz, 1H), 5.44 (dd, J=7.9 Hz, J=4.6 Hz, 1H), 5.62 (dd, J=7.9 Hz, J=7.0 Hz, 1H), 6.37 (d, J=4.6 Hz, 1H).

### Example 2

### Preparation and characterization of crystalline 1,2,3,6-tetrapivaloyl-α-L-altrofuranose (III)

A solution of pyridine (3.82 kg) in methylene chloride (15 L) was cooled to 0° C. under nitrogen atmosphere. Trifluoromethanesulfonic anhydride (3.28 kg) was added dropwise at 0° C., followed by dropwise addition of 1,2,3,6-tetrapivaloyl-α-D-galactofuranoside (5 kg) solution in methylene chloride (10 L). The reaction mixture was stirred at 0° C. for 2 hours and reaction was checked for completion by TLC (hexane:ethyl acetate =4:1). If reaction was not complete at this point, additional portion of trifluoromethanesulfonic anhydride (0.1 kg) was added. A triflated compound 5-trifluoromethanesulfonyloxy-5-deoxy-1,2,3,6-tetrapivaloyl-α-D-galactofuranoside was formed from the galactofuranoside at this stage in the reaction. The reaction mixture was subsequently washed with cold 6% hydrochloric acid (3 times 30 L), brine (30 L) and 7.5% sodium bicarbonate solution (30 L). N,N-diisopropylethylamine (230 mL) was then added and reaction was stirred over sodium carbonate (1.5 kg) for 1 hour. The reaction was filtered off and concentrated to dryness. Essentially pure 5-trifluoromethanesulfonyloxy-5-deoxy-1,2,3,6-tetrapivaloyl-α-D-galactofuranose was isolated as crystalline solid.

5-trifluoromethanesulfonyloxy-5-deoxy- 1,2,3,6-tetrapivaloyl-α-D-galactofuranose was dissolved in 9.5 L of DMF, and reacted with 5 equivalents (1.67 kg) of sodium nitrite for 12 hours. The reaction was diluted with heptane (24 L) and ethyl acetate (12 L), filtered off and poured into a 2% bicarbonate solution (40 L). The product was extracted with heptane/ethyl acetate and crystallized from heptane as done in Example 1. The yield of 1,2,3,6-tetrapivaloyl-α-L-altrofuranoside (III) was 35-45% (2 kg from 5 kg of (II). HPLC demonstrated the complete conversion to the inverted alcohol. Product was off-white crystalline solid. M.P. 109-112° C., IR (KBr, cm⁻¹): 3444 (OH, s), 2977 (C—H stretch, s), 1732 (ester of pivalate, vs),1481 (weak), 1284 (C—O, weak), 1156 (C—O, vs), 1028 (C—O, weak); ¹H NMR (CDCl₃, 400 MHz, TMS): δ=1.18 (s, 3H), 1.20 (s, 3H), 1.21 (s, 3H), 1.22 (s, 3H), 3.01 (d, J=2.45, 1H), 3.99-4.02 (m, 2H), 4.11-4.07 (m, 1H), 4.26 (dd, J=12.4 Hz, J=2.7 Hz, 1H), 5.43 (dd, J=7.3 Hz, J=4.6 Hz, 1H), 5.58 (dd, J=7.3 Hz, J=5.2 Hz, 1H), 6.37 (d, J=4.8 Hz, 1H).

### Example 3

### Preparation and characterization of crystalline 5-azido-5-deoxy-1,2,3,6-tetrapivaloyl-α-D-galactofuranose (IV)

A triflated compound, 5-trifluoromethanesulfonyloxy-5-deoxy-1,2,3,6-tetrapivaloyl -α-L-altrofuranose, was formed from the altrofuranose III (5 kg) of Example 2 in the procedure as described in Example 2. This compound was reacted with sodium azide (1.6 kg) in DMF (9.5 L). The reaction was performed using the optimum conditions observed during an inversion reaction. The crude product was crystallized twice from methanol (1.3-1.7 mL/g). On 5 kg scale, the yield of 5-azido-5-deoxy-1,2,3,6-tetrapivaloyl-α-D-galactofuranose (IV) from III was usually 65-70% (˜3.3 kg). Product was white crystalline solid. M.P. 103-104° C. IR (KBr, cm⁻¹): 2090 (azide, s), 1740 (ester of pivalate, vs),1480 (weak), 1280 (C—O, s), 1160 (C—O, vs), 1042 (C—O, weak); ¹H NMR (CDCl₃, 400 MHz, TMS): δ=1.19 (s, 3H), 1.20 (s, 3H), 1.22 (s, 3H), 1.25 (s, 3H), 3.83-3.79 (m, 1H), 4.05 (dd, J=6.7, J=4.8 Hz 1H), 4.15 (dd, J=11.7 Hz, J=8.0 Hz, 1H), 4.30 (dd, J=11.7 Hz, J=4.2 Hz, 1H), 5.41 (dd, J=7.9 Hz, J=4.6 Hz, 1H), 5.59 (t, J=7.5 Hz, 1H), 6.33 (d, J=4.5 Hz, 1H).

### Example 4

### Crystalline 5-azido-5-deoxy-1,2,3,6-tetrapivaloyl-α-D-galactofuranose (IV)

The crude product formed in Example 3 was crystallized from EtOAc:MeOH 1:6 and methanol using the crystallization procedure described above. The yield for this crystallization was 50-60% 5-azido-5-deoxy-1,2,3,6-tetrapivaloyl-α-D-galactofuranose (IV).

### Example 5

### Preparation of crystalline 5-benzylthio-5-deoxy-1,2,3,6-tetrapivaloyl-α-D-galactofuranoside

5-Benzylthio-5-deoxy-1,2,3,6-tetrapivaloyl-α-D-galactofuranose is prepared in a similar manner as 5-azido-5-deoxy-1,2,3,6-tetrapivaloyl-α-D-galactofuranose by replacing the sodium azide with sodium a-toluenethioxide and crystallizing the sample as described in Example 3.

Many variations of the present invention will suggest themselves to those skilled in the art in light of the above detailed description. For example, the crystallization of the sugar may be performed from various solvents. All such obvious variations are within the fully intended scope of the appended claims. Those of skill in the art should, in light of the present disclosure, appreciate that many changes can be made in the specific embodiments where are disclosed herein and still obtain a like or similar result without departing from the spirit and scope of the invention.

The above mentioned patents, applications, test methods, publications are hereby incorporated by reference their entirety.

What is claimed is:

1. A crystalline furanose of the formula: 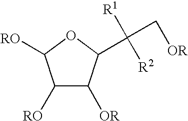
wherein each R is independently H, acetyl, methylacetyl, dimethylacetyl, trimethylacetyl, or a protecting group, and at least two Rs are selected from the group consisting of methylacetyl, dimethylacetyl, and trimethylacetyl;
R¹ and R² are independently H, OH, OR³, N₃, NH₂, NHR³, NR³₂, SH, SR³, OS(═O)₂R³, C(═O)R³, methylacetoxy, dimethylacetoxy, trimethylacetoxy, acetoxy, chloroacetoxy, dichloroacetoxy, trichloroacetoxy or an O-protecting group, wherein at least one of R¹ and R² is H; and
each R³ is independently H or a substituted or unsubstituted C₁-C₁₂ alkyl, C₂-C₁₂ alkenyl, C₂-C₁₂ alkynyl, C₅-C₆ cycloalkyl, C₅-C₁₂ cycloalkenyl, C₅-C₁₂ aryl, C₄-C₁₂ heteroaryl, C₆-C₁₂ arylalkyl, C₄-C₁₂ heterocycle, C₆-C₁₂ heterocycloalkyl, C₅-C₁₂ heteroarylalkyl, a C₂-C₁₂ acyl, or a combination thereof; and
wherein the molecular weight of the furanose is between 300 g/mol and 1000 g/mol.

2. The crystalline furanose of claim 1, wherein the furanose has a molecular weight of at least 350 g/mol.

3. The crystalline furanose of claim 2, wherein the furanose has a molecular weight of at least 400 g/mol.

4. The crystalline furanose of claim 3, wherein furanose has a molecular weight of at least 450 g/mol.

5. The crystalline furanose of claim 1, wherein at least three R groups are trimethylacetyl.

6. The crystalline furanose of claim 5, wherein the furanose is a tetrapivaloyl furanose.

7. The crystalline furanose of claim 1, wherein R¹ is OH or N₃ and R² is H.

8. The crystalline furanose of claim 1, wherein the furanose is 1,2,3,6-tetrapivaloyl-α-D-galactofuranose or 1,2,3,6-tetrapivaloyl-α-L-altrofuranose.

9. The crystalline furanose of claim 1, wherein the furanose is 5-azido-5-deoxy-1,2,3,6-tetrapivaloyl-α-D-galactofuranose.

10. A method for producing a crystalline furanose represented by the formula: 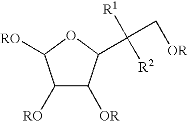
wherein each R is independently H, acetyl, methylacetyl, dimethylacetyl, trimethylacetyl, or a protecting group, and at least two Rs are selected from the group consisting of methylacetyl, dimethylacetyl, and trimethylacetyl;
R¹ and R² are H, OH, OR³, N₃, NH₂, NHR³, NR³₂, SH, SR³, OS(═O)₂R³, C(═O)R³, methylacetoxy, dimethylacetoxy, trimethylacetoxy, acetoxy, chloroacetoxy, dichloroacetoxy, trichloroacetoxy or an O-protecting group, wherein at least one of R¹ and R² is H;
each R³ is independently H or a substituted or unsubstituted C₁-C₁₂ alkyl, C₂-C₁₂ alkenyl, C₂-C₁₂ alkynyl, C₅-C₆ cycloalkyl, C₅-C₁₂ cycloalkenyl, C₅-C₁₂ aryl, C₄-C₁₂ heteroaryl, C₆-C₁₂ arylalkyl, C₄-C₁₂ heterocycle, C₆- C₁₂ heterocycloalkyl, C₅-C₁₂ heteroarylalkyl, a C₂-C₁₂ acyl, or a combination thereof; and
wherein the molecular weight of the furanose is between 300 g/mol and 1000 g/mol,
comprising adding the furanose to, or forming the furanose in a solvent; and crystallizing the furanose from the solvent.

11. The method of claim 10, wherein the furanose has a molecular weight of at least 350 g/mol.

12. The method of claim 11, wherein the furanose has a molecular weight of at least 400 g/mol.

13. The method of claim 12, wherein furanose has a molecular weight of at least 450 g/mol.

14. The method of claim 10, wherein at least three R groups are trimethylacetyl.

15. The method of claim 16, wherein furanose is a tetrapivaloyl furanose.

16. The method of claim 15, wherein at least one of monopivaloyl, dipivaloyl, tripivaloyl, or pentapivaloyl furanose is formed in addition to the tetrapivaloyl furanose, and the monopivaloyl, dipivaloyl, tripivaloyl, or pentapivaloyl furanose is not crystallized when the tetrapivaloyl furanose is crystallized.

17. The method of claim 10, wherein R¹ is OH and R² is H.

18. The method of claim 17, wherein the solvent comprises heptane.

19. The method of claim 10, wherein R¹ is N₃ and R² is H.

20. The method of claim 19, wherein the solvent comprises methanol.

21. The method of claim 10, wherein crystallizing comprises cooling the solvent system, allowing the solution to cool without an external cooling source, adding a seed crystal, adding an additional solvent or solvent system to cause the furanose to precipitate out of solution, or a combination thereof.

22. The method of claim 21, wherein crystallizing comprises first heating the furanose and the solvent to a temperature near the boiling point of the solvent, and then cooling to a temperature of between −20° C. and −10° C., and waiting for at least 36 hours.

23. The method of claim 10, wherein the method further comprises:
adding a second solvent, wherein the second solvent is miscible with the solvent and capable of dissolving the furanose; and
subjecting the solution to a crystallization treatment, to obtain said crystalline form of the furanose.
